(19) United States
(12) Patent Application Publication
(10) Pub. No.: US 2007/0093433 A1
(43) Pub. Date: Apr. 26, 2007

(54) Aminoglycoside antibiotics for use as vap-1/ssao inhibitors

(76) Inventors: Sirpa Jalkanen, Piispanristi (FI); Marko Salmi, Turku (FI); Markku Jalkanen, Piispanristi (FI)

Correspondence Address: ROTHWELL, FIGG, ERNST & MANBECK, P.C., 1425 K STREET, N.W., SUITE 800, WASHINGTON, DC, 20005, US

(21) Appl. No.: 10/579,791

(22) PCT Filed: Dec. 17, 2004
(86) PCT No.: PCT/FI04/00772

§ 371 Date: May 18, 2006

(30) Foreign Application Priority Data
Dec. 31, 2003 (FI) 20031927

(51) Int. Cl. A61K 31/7034

(52) U.S. Cl. 514/35

(57) ABSTRACT

The present invention concerns the use of a compound comprising one or more sugar moieties, which optionally are aminosubstitutes, and possibly other moieties, wherein said compound is a molecule comprising at least two aminosubstituents, said aminosubstituents being primary, secondary or tertiary amino groups, wherein said aminosubstituents are either attached to one single sugar moiety or attached to several sugar moieties or other moieties of the molecule, or to chains connecting two moieties or to chains being substituents to the molecule, for the manufacture of a pharmaceutical preparation useful as an agent capable of influencing an amine oxidase enzyme activity, more particularly inhibiting VAP-1/SSAO activity. The preferred compounds are aminoglycoside antibiotics, e.g., streptomycin, netilmicin, geneticin, gentamicin, puromycin, tobramycin and amikacin. The preferred conditions to be treated are inflammatory diseases or conditions, diseases related to carbohydrate metabolism, diseases related to aberrations in adipocyte differentiation or function or smooth muscle cell function, and vascular diseases.

### FIELD OF THE INVENTION

The present invention is directed to polyaminosubstituted sugars being capable of influencing copper-containing amine oxidases commonly known as semicarbazide-sensitive amine oxidases (SSAO), including the human SSAO known as Vascular Adhesion Protein-I (VAP-1). These polyaminosubstituted sugars are SSAO inhibitors having therapeutic utility as drugs to treat or prevent conditions and diseases such as a number of inflammatory conditions and diseases related to carbohydrate metabolism and to aberrations in adipocyte differentiation or function and smooth muscle cell function, and vascular diseases.

### BACKGROUND OF THE INVENTION

The publications and other materials used herein to illuminate the background of the invention, and in particular, cases to provide additional details respecting the practice, are incorporated by reference.

VAP-1 is a human endothelial cell adhesion molecule that has several unique properties that distinguish it from the other inflammation-related adhesion molecules. It has a unique and restricted expression pattern and mediates lymphocyte binding to vascular endothelium (Salmi, M., and Jalkanen, S., Science 257:1407-1409 (1992)). Inflammation induces the upregulation of VAP-1 to the surface of vascular endothelial cells mediating leukocyte entry to skin, gut and inflamed synovium (Salmi, M., and Jalkanen, S., Science 257:1407-1409 (1992); Salmi, M, et al., J. Exp. Med 178:2255-2260 (1993); Arvillommi, A., et al., Eur. J. Immunol 26:825-833 (1996); Salmi, M., et al., J. Clin. Invest. 99:2165-2172 (1997): (Salmi. M., and Jalkanen, S., J. Exp. Med. 183:569-579 (1996); J. Exp. Med 186:589-600 (1997)). One of the most interesting features of VAP-1 is a catalytic extracellular domain which contains a monoamine oxidase activity (Smith, D. J., et al., J. Exp. Med 188:17-27 (1998)).

The cloning and sequencing of the human VAP-1 cDNA revealed that it encodes a transmembrane protein with homology to a class of enzymes called the copper-containing amine oxidases (E.C. 1.4.3.6). Enzyme assays have shown that VAP-1 possesses a monoamine oxidase (MAO) activity which is present in the extracellular domain of the protein (Smith, D. J., et al., J. Exp. Med. 188:17-27 (1998)). Thus, VAP-1 is an ecto-enzyme. Analysis of the VAP-1 MAO activity showed that VAP-1 belongs to the class of membrane-bound MAO's termed semicarbazide-sensitive amine oxidases (SSAO). These are distinguished from the widely distributed mitochondrial MAO-A and B flavoproteins by amino acid sequence, cofactor, substrate specificity and sensitivity to certain inhibitors. However, certain substrates and inhibitors are common to both SSAO and MAO activities. The mammalian SSAO's can metabolize various monoamines produced endogenously or absorbed as dietary or xenobiotic substances. They act principally on primary aliphatic or aromatic monoamines such as methylamine or benzylamine (Lyles G. A., Int. J.

Biochem. Cell Biol, 28:259-274 (1996)). Thus, VAP-1 located on the vascular endothelial cell surface can act on circulating primary monoamines with the following reaction pathway.
RNH₂+O₂+H₂O------->RCHO+H₂O₂+NH₃

The physiological substrates of VAP-1 SSAO in man have not been clearly identified. However, methylamine is a good substrate for VAP-1 SSAO. Methylamine is a product of various human biochemical pathways for the degradation of creatinine, sarcosine and adrenaline, and is found in various mammalian tissues and in blood. It can also be derived from the diet by gut bacterial degradation of dietary precursors. The concentration of methylamine in the blood can be increased in certain physiological and pathological situations such as diabetes. Another potential physiological substrate is aminoacetone.

VAP-1 SSAO activity has been proposed to be directly involved in the pathway of leukocyte adhesion to endothelial cells by a novel mechanism involving direct interaction with an amine substrate presented on a VAP-1 ligand expressed on the surface of a leukocyte (Salmi et al. Immunity, (2001)). This publication describes the direct involvement of VAP-1 SSAO activity in the process of adhesion of leukocytes to endothelium. Thus inhibitors of VAP-1 SSAO activity could be expected to reduce leukocyte adhesion in areas of inflammation and thereby reduce leukocyte trafficking into the inflamed region and therefore the inflammatory process itself.

In human clinical tissue samples expression of VAP-1 is induced at sites of inflammation. This increased level of VAP-1 can lead to increased production of H₂O₂ generated from the action of the VAP-1 SSAO extracellular domain on monoamines present in the blood. This generation of H₂O₂ in the localized environment of the endothelial cell could initiate other cellular events. H₂O₂ is a known signaling molecule that can upregulate other adhesion molecules and this increased adhesion molecule expression may lead to enhanced leukocyte trafficking into areas in which VAP-1 is expressed. It also may be that other products of the VAP-1 SSAO reaction could have biological effects also contributing to the inflammatory process. Thus the products of the VAP-1 SSAO activity may be involved in an escalation of the inflammatory process which could be blocked by specific SSAO inhibitors.

VAP-1 SSAO may be involved in a number of other pathological conditions associated with an increased level of circulating amine substrates of VAP-1 SSAO. The oxidative deamination of these substrates would lead to an increase in the level of toxic aldehydes and oxygen radicals in the local environment of the endothelial cell which could damage the cells leading to vascular damage. Increased levels of methylamine and aminoacetone have been reported in patients with Type I and Type II diabetes and it has been proposed that the vasculopathies such as retinopathy, neuropathy and nephropathy seen in late stage diabetes could be treated with specific inhibitors of SSAO activity.

Takahashi, H, et al., Yakugaku Zasshi 101(12): 1154-1156 (1981) report the synthesis of a number of N-alkylaminoephedrines, including N-(isopropylideneamino)-ephedrine or R,S-(+)-(2-hydroxy-1-methyl-2-phenylethyl)methylhydrazone-2-propanone. These hydrazone compounds were synthesized to evaluate their effect on the bronchial musculature and were found not to exhibit any significant activity.

Grifantini, M., et al., Farmaco, Ed. Sci.23(3):197-203 (1968), report the synthesis of several alkyl- and acyl-derivatives of N-amino-1-ephedrine and N-amino-d-pseudoephedrine having antidepressant and monoamine oxidase inhibitory properties.

Jeffrey O'Sullivan et al., Biochimica et Biophysica Acta 1647 (2003) 367-371 report the inhibition of semicarbazide-sensitive amine oxidases by certain aminohexoses, namely glucosamine, galactosamine and mannosamine. These compounds are all monoaminosubstituted.

The international patent publications WO 02/020290 and WO 03/006003 disclose certain hydrazino compounds useful as specific VAP-1 SSAO inhibitors that modulate VAP-1 activity. These compounds are described as useful for the treatment of acute and chronic inflammatory conditions or diseases as well as diseases related to carbohydrate metabolism, aberrations in adipocyte differentiation or function and smooth muscle cell function, and various vascular diseases.

### OBJECTS AND SUMMARY OF THE INVENTION

VAP-1/SSAO catalyzes oxidative deamination of amines in a reaction which results in the production of the corresponding aldehyde, hydrogen peroxide and ammonium. The reaction products are pro-inflammatory compounds. Thus, inhibition of the enzymatic activity of VAP-1 results in diminished production of these pro-inflammatory substances and thus has anti-inflammatory effects.

The object of the present invention is to provide the use of polyaminosubstituted sugars as agents capable of inhibiting amine oxidase activity.

Thus, this invention concerns the use of a compound comprising one or more sugar moieties, which optionally are aminosubstitutes, and possibly other moieties, wherein said compound is a molecule comprising at least two aminosubstituents, said aminosubstituents being primary, secondary or tertiary amino groups, wherein said aminosubstituents are either attached to one single sugar moiety or attached to several sugar moieties or other moieties of the molecule, or to chains connecting two moieties or to chains being substituents to the molecule, for the manufacture of a pharmaceutical preparation useful as an agent capable of influencing an amine oxidase enzyme activity.

### DETAILED DESCRIPTION OF THE INVENTION

Definitions:

The term “treatment” or “treating” shall be understood to include complete curing of a disease or condition, as well as amelioration or alleviation of said disease or condition.

The term “prevention” shall be understood to include complete prevention, prophylaxis, as well as lowering the individual's risk of falling ill with said disease or condition.

The term “individual” refers to a human or animal subject.

The term “compound” shall here be understood to cover any geometric isomer, stereoisomer, diastereoisomer, racemate or any mixture of isomers, and any pharmaceutically acceptable salt of said compound.

“Moiety” shall be understood as a ring or ring system.

Preferable Embodiments:

The polyaminosubstituted compounds for use according to this invention, can according to one embodiment, be compounds consisting of a single sugar unit (moiety). It may, however be preferable to have also other ring units and/or additional sugar units in the molecule, just in order to provide molecules with a high degree of amino substitution.

The sugar unit is preferable a hexose such as glucose, mannose, galactose, fructose or sorbose, or a pentose such as arabinose, xylose, ribose, rhamnose or fucose.

In case the molecule comprises several sugar units, these can be the same or different sugars.

According to a preferable embodiment, the aminosubstituents are primary amino substituents (NH₂-groups) either attached to one single sugar moiety or attached to several sugar moieties or other moieties of the molecule.

In one preferable aspect, the molecule is an oligosaccaride, preferable a disaccaride, such as sucrose, maltose or lactose.

The sugar unit(s) of the molecule can also be substituted with other substituents in addition to the aminosubstituents.

According to another aspect, the molecule is a glycoside, i.e. a compound formed by a reaction of a hydroxyl group of a sugar unit with a hydroxyl group of another compound such as a non-sugar compound, where such a non-sugar compound preferably is a compound comprising one or more rings.

According to an especially preferable embodiment, the compound is an aminoglycoside, particularly an aminoglycoside antibiotic.

Aminoglycoside antibiotics are widely used for treating infections. However, no use of this group of compounds for treating or preventing non-infectious inflammatory conditions has been disclosed or suggested in the art.

Preferable amine oxidase inhibitors:

As examples of powerful inhibitors can be mentioned aminoglycoside antibiotics such as the compounds shown in Scheme 1.

According to one important aspect, the invention concerns the use of a compound active as an amine oxidase inhibitor for the manufacture of a pharmaceutical preparation for treatment or prevention of any disease or condition benefiting from inhibiting an amine oxidase enzyme.

Diseases or conditions with responsiveness to amine oxidase inhibitors:

As examples of groups of diseases or conditions the treatment or prevention of which would benefit from inhibiting amine oxidase enzyme can be mentioned inflammatory diseases or conditions; diseases related to carbohydrate metabolism; diseases related to aberrations in adipocyte differentiation or function or smooth muscle cell function and vascular diseases. However, the diseases or conditions are not restricted to these groups.

According to one embodiment, the inflammatory disease or condition can be a connective tissue inflammatory disease or condition, such as, but not limited to ankylosing spondylitis, Reiter's syndrome, psoriatic arthritis, osteoarthritis or degenerative joint disease, rheumatoid arthritis, Sjögren's syndrome, Bechet's syndrome, relapsing polychondritis, systemic lupus erythematosus, discoid lupus erythematosus, systemic sclerosis, eosinophilic fasciitis, polymyositis and dermatomyositis, polymyalgia rheumatica, vasculitis, temporal arteritis, polyarterisis nodosa, Wegner's granulamatosis, mixed connective tissue disease, or juvenile rheumatoid arthritis.

According to another embodiment, said inflammatory disease or condition is a gastrointestinal inflammatory disease or condition, such as, but not limited to Crohn's disease, ulcerative colitis, irritable bowel syndrome (spastic colon), fibrotic conditions of the liver, inflammation of the oral mucosa (stomatitis), or recurrent aphtous stomatitis.

According to a third embodiment, said inflammatory disease or condition is a central nervous system inflammatory disease or condition, such as, but not limited to multiple sclerosis, Alzheimer's disease, or ischemia-reperfusion injury associated with ischemic stroke.

According to a fourth embodiment, said inflammatory disease or condition is a pulmonary inflammatory disease or condition, such as, but not limited to asthma, chronic obstructive pulmonary disease, or adult respiratory distress syndrome.

According to a fifth embodiment, said inflammatory disease or condition is a skin inflammatory disease or condition such as, but not limited to contact dermatitis, atopic dermatitis, psoriasis, pityriasis rosea, lichen planus, or pityriasis rubra pilaris.

According to a seventh embodiment said inflammatory condition is related to tissue trauma or resulting from organ transplantations or other surgical operations.

According to an eighth embodiment, said disease related to carbohydrate metabolism is a disease such as but not limited to diabetes, atherosclerosis, vascular retinopathies, retinopathy, nephropathy, nephrotic syndrome, polyneuropathy, mononeuropathies, autonomic neuropathy, foot ulcers or joint problems.

According to a tenth embodiment said disease relating to aberrations in adipocyte differentiation or function or smooth muscle cell function is a disease such as but not limited to atherosclerosis or obesity.

According to an eleventh embodiment, the vascular disease is a disease such as but not limited to atheromatous ateriosclerosis, nonatheromateous ateriosclerosis, ischemic heart disease, peripheral aterial occlusion, thromboangiitis obliterans (Buerger's disease), or Raynaud's disease and phenomenon.

For the purpose of this invention, the compounds disclosed in this invention or their isomer, isomer mixture or their pharmaceutically acceptable salts can be administered by various routes. For example, administration can be by parenteral, subcutaneous, intravenous, intraarticular, intrathecal, intramuscular, intraperitoneal, or intradermal injections, or by transdermal, buccal, oromucosal, ocular routes or via inhalation. Alternatively, or concurrently, administration can be by the oral route. Particularly preferred is oral administration. Suitable oral formulations include e.g. conventional or slow-release tablets and gelatine capsules.

The required dosage of the compounds will vary with the particular disease or condition being treated, the severity of the condition, the duration of the treatment, the administration route and the specific compound being employed.

Thus, a typical dose is in the dosage range of about 0.1 microgram/kg to about 300 mg/kg, preferably between 1.0 microgram/kg to 10 mg/kg body weight. Compounds of the present invention may be administered in a single daily dose, or the total daily dosage may be administered in divided doses of two, three or four times daily.

The invention will be illuminated by the following non-restrictive Experimental Section.

### EXPERIMENTAL SECTION

Enzymatic Assays

Radiochemical Measurements of Monoamine Oxidase Activity

Amine oxidase activity was assayed radiochemically using [7-¹⁴C]-benzylamine hydrochloride (spec. act. 57 mCi/mmol, Amersham) as a substrate. In brief, the cells (VAP-1 transfected Ax endothelial cells or VAP-1 transfected CHO cells and their mock transfected controls) were seeded onto gelatin-coated 24-well tissue culture plates and allowed to reach confluence. Prior to experiments, the cells were rinsed twice with RPMI 1640 and pre-incubated 30 min at 37° C. in 0.3 ml RPMI-1640 medium containing amikacin, tobramycin, gentamicin, streptomicin or geneticin (1 mg/ml). The reaction was initiated by addition of 6 μmol/L [¹⁴C]-benzylamine (40000 dpm) and terminated after 1 hour by citric acid. The aldehydes were extracted into toluene containing diphenyloxazole and the formation of [¹⁴C]-labelled benzaldehyde was quantified by scintillation counting.

Fluorometric Detection of SSAO-mediated H₂O₂ Formation

SSAO activity of the cells was also independently measured using Amplex Red reagent (10-acetyl-3,7-dihydroxyphenoxazine; Molecular Probes Europe BV), a highly sensitive and stable probe for H₂O₂. Cultured cells (VAP-1 transfected Ax endothelial cells or VAP-1 transfected CHO cells and their mock transfected controls) were rinsed with Krebs Ringer phosphate glucose (KRPG; 145 mM NaCl, 5.7 mM sodium phosphate, 4.86 mM KCl, 0.54 mM CaCl₂, 1.22 mM MgSO₄, 5.5 mM glucose, pH 7.35) and pre-incubated 30 min at 37° C. in 200 μl KRPG containing amikacin, tobramycin, gentamicin, netilmicin, streptomycin, geneticin, glucosamine, mannosamine, galactosamine, or puromycin (1 mg/ml and 100 μg/ml). Catalytic reaction was initiated by addition of benzylamine as substrates and H₂O₂-detecting mixture containing horseradish peroxidase (final concentration 0.8 U/ml) and Amplex Red reagent (60 μM). The plates were incubated for 1-2 hours at 37° C. in the final volume of 250 μl, the bathing medium was clarified by centrifugation and placed in aliquots (200 μl) into white non-phosphorescent microplates (Cliniplate). Fluorescence intensity of the samples was measured (excitation, 545 nm; emission, 590 nm; Tecan ULTRA fluoropolarometer) and H₂O₂ concentration was calculated from calibration curves generated by serial dilutions of either standard H₂O₂ or resorufin, the product of the Amplex Red reaction (Molecular Probes).

Results

The representative inhibitory percentages of the different agents obtained in 2 to 5 experiments is presented in the following table. The monoamine compounds glucosamine, galactosamine and mannosamine were tested as reference compounds.

TABLE I

| Agent | % of inhibition |
|---|---|
| Glucosamine | 0% |
| Galactosamine | 12% |
| Mannosamine | 20% |
| Streptomicin | 5% |
| Netilmicin | 45% |
| Geneticin | 46% |
| Gentamicin | 46% |
| Puromycin | 59% |
| Tobramycin | 78% |
| Amikacin | 84% |

SSAO activity of the cells was entirely dependent of the transfected VAP-1. Its enzymatic activity was diminished to variable extent by netilmicin, gentamicin, geneticin, puromycin, tobramycin and amikacin. In contrast, monoamino hexoses (i.e. the reference compounds glucosamine, galactosamine and mannosamine) and other agents tested did not significantly inhibit the activity of VAP-1 (Table I).
Aminoglycosides Bind to VAP-1 Also In Vivo
Generation of mTIEhVAP-1 Transgenic/VAP-1 Knockout Mice (VAP KO+TG) and Their Use to Test, Whether Human VAP-1 Binds Aminoglycosides In Vivo

The mTIEhVAP-1 line E35 mice expressing human VAP-1 on vasculature were crossed to VAP-1 knockout mice that were previously created by using conventional gene targeting techniques to replace the mouse VAP-1 gene with a nonfunctional mutant-allele. The mTIEhVAP-1 transgene, mouse VAP-1 mutant-allele and endogenous mouse VAP-1 allele were all identified by PCR screening of purified genomic DNA with specific primers and verified immunohistochemically with human and mouse VAP-1 antibodies.

These mice and VAP KO mice as controls received intravenously tobramycin 3 mg/kg and tobramycin concentration from serum was measured after 30 min, 1, 2 and 3 hours after injections using fluorescence polarization immunoassay.

Results

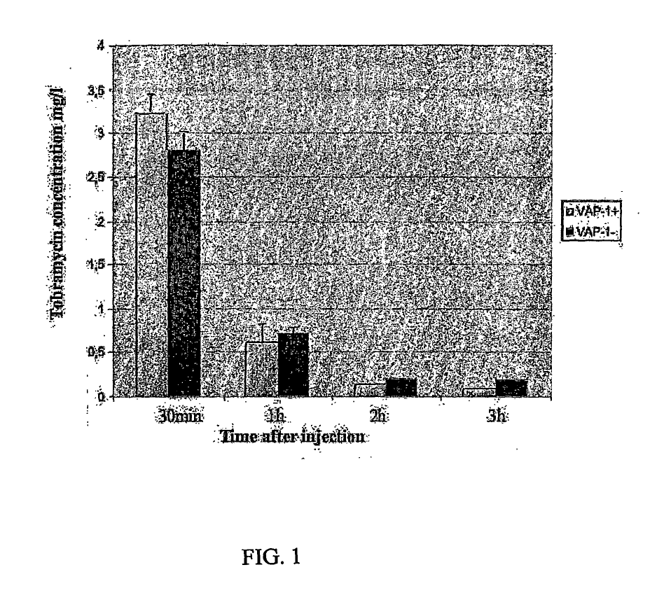

The results are illustrated in FIG. 1, which shows tobramycin concentration after intravenous injections of 3 mg/kg of tobramycin. VAP-1+ indicates VAP KO+TG mice and VAP-1− are VAP KO mice. The concentration of the tobramycin decreased gradually in the serum in both groups but at later time points (at 2 and 3 hours) the VAP KO+VAP-1 had lower concentrations of tobramycin in the serum than VAP KO mice. At 3 hours the difference was two fold, FIG. 1. These findings strongly suggest that tobramycin binds to human VAP-1 also in vivo, and the tobramycin bound to endothelial VAP-1 in vessels accounts for the reduced concentration in the circulating blood.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

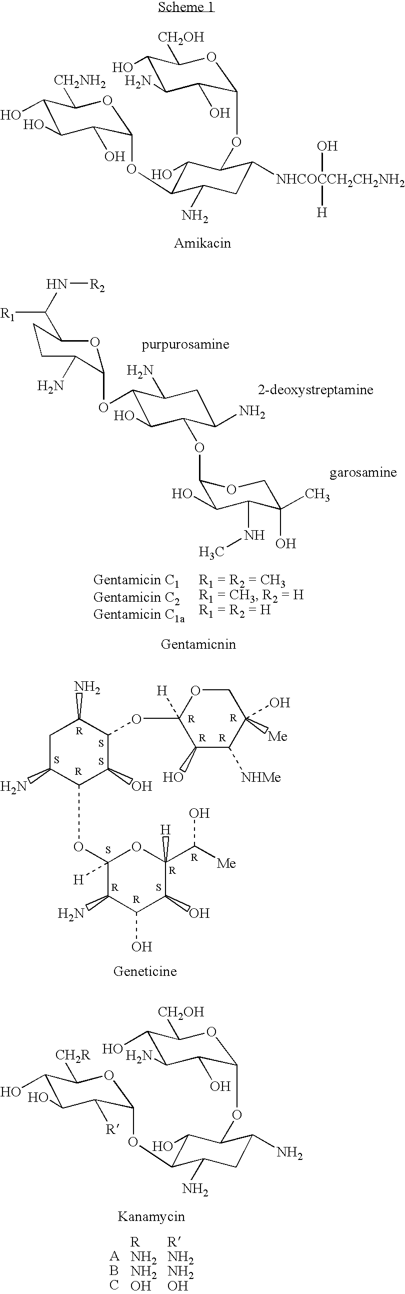

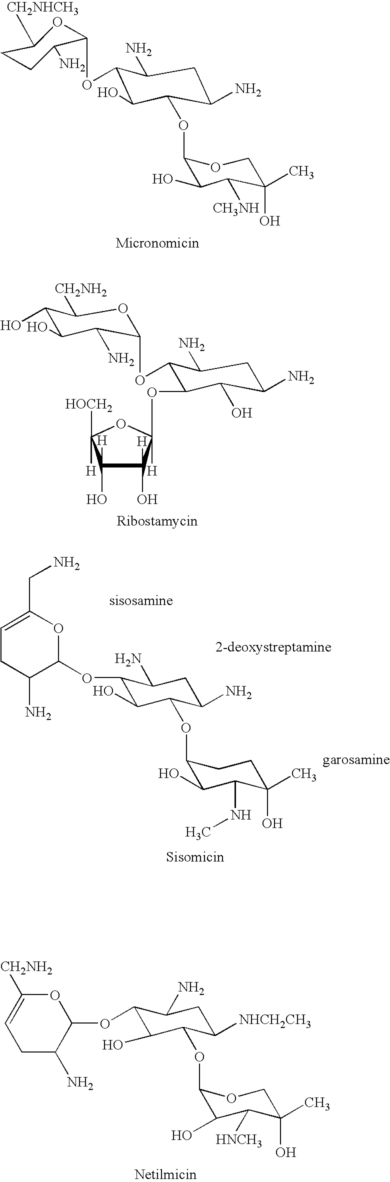

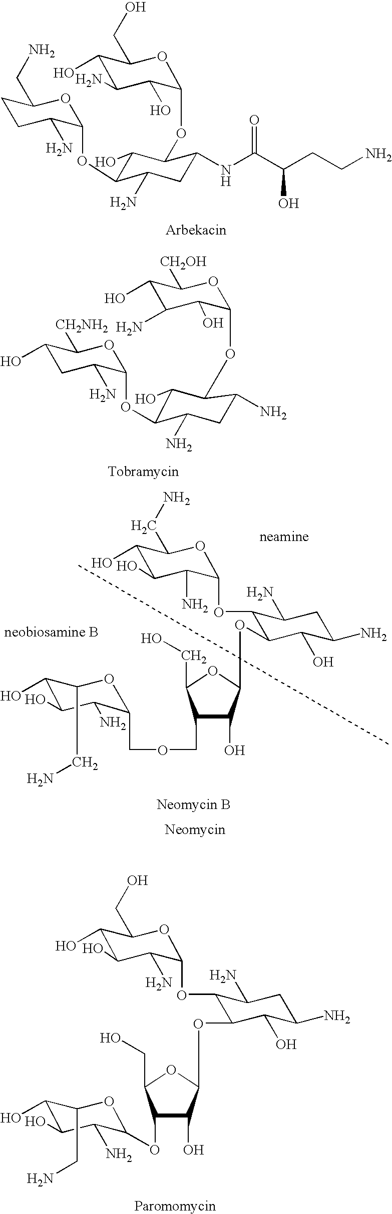

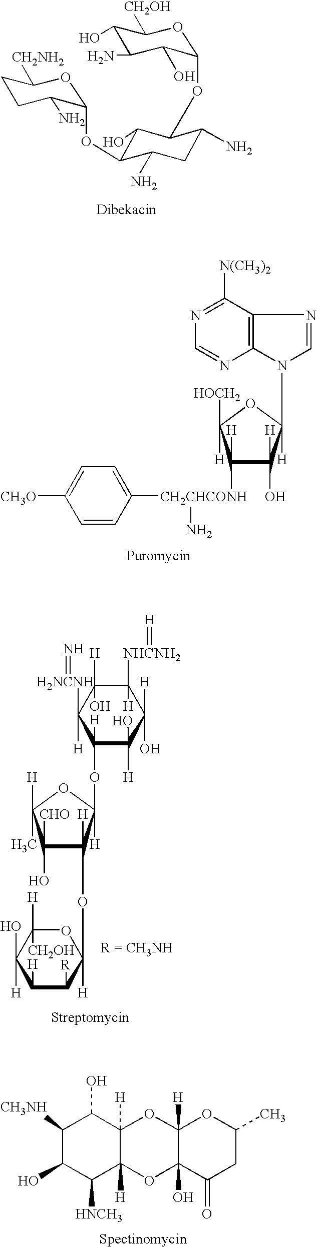

1-19. (canceled)

20. A method for influencing an amine oxidase enzyme activity in an individual, said method comprising administering to said individual a compound comprising one or more sugar moieties, which optionally are aminosubstitutes, and possibly other moieties, wherein said compound is a molecule comprising at least two aminosubstituents, said aminosubstituents being primary, secondary or tertiary amino groups, wherein said aminosubstituents are either attached to one single sugar moiety or attached to several sugar moieties or other moieties of the molecule, or to chains connecting two moieties or to chains being substituents to the molecule.

21. The method according to claim 20, wherein the aminosubstituents are primary amino substituents (NH₂-groups) either attached to one single sugar moiety or attached to several sugar moieties or other moieties of the molecule.

22. The method according to claim 20 wherein the molecule comprises several sugar moieties and/or other moieties.

23. The method according to claim 20, wherein the compound is an aminoglycoside, especially an aminoglycoside antibiotic.

24. A method for treatment or prevention of a disease or condition in an individual, benefiting from inhibiting an amine oxidase enzyme, wherein the disease or condition is an inflammatory disease or condition; a disease related to carbohydrate metabolism; a disease related to aberrations in adipocyte differentiation or function or smooth muscle cell function; or a vascular disease, said method comprising administering of a compound as defined in claim 1 to said individual.

25. The method according to claim 24 wherein said inflammatory disease or condition is a connective tissue inflammatory disease or condition.

26. The method according to claim 25, wherein said connective tissue inflammatory disease or condition is selected from the group consisting of ankylosing spondylitis, Reiter's syndrome, psoriatic arthritis, osteoarthritis or degenerative joint disease, rheumatoid arthritis, Sjögren's syndrome, Bechet's syndrome, relapsing polychondritis, systemic lupus erythematosus, discoid lupus erythematosus, systemic sclerosis, eosinophilic fasciitis, polymyositis and dermatomyositis, polymyalgia rheumatica, vasculitis, temporal arteritis, polyarterisis nodosa, Wegner's granulamatosis, mixed connective tissue disease, and juvenile rheumatoid arthritis.

27. The method according to claim 24 wherein said inflammatory disease or condition is a gastrointestinal inflammatory disease or condition.

28. The method according to claim 27 wherein the gastrointestinal inflammatory disease or condition is selected from the group consisting of Crohn's disease, ulcerative colitis, irritable bowel syndrome (spastic colon), fibrotic conditions of the liver, inflammation of the oral mucosa (stomatitis), and recurrent aphtous stomatitis.

29. The method according to claim 24 wherein said inflammatory disease or condition is a central nervous system inflammatory disease or condition.

30. The method according to claim 29, wherein said central nervous system inflammatory disease or condition is selected from the group consisting of multiple sclerosis, Alzheimer's disease, and ischemia-reperfusion injury associated with ischemic stroke.

31. The method according to claim 24 wherein said inflammatory disease or condition is a pulmonary inflammatory disease or condition.

32. The method according to claim 31, wherein said pulmonary inflammation disease or condition is selected from the group consisting of asthma, chronic obstructive pulmonary disease, and adult respiratory distress syndrome.

33. The method according to claim 24, wherein said inflammatory disease or condition is a skin inflammatory disease or condition.

34. The method according to claim 33, wherein said skin inflammatory disease or condition is selected from the group consisting of contact dermatitis, atopic dermatitis, psoriasis, pityriasis rosea, lichen planus, and pityriasis rubra pilaris.

35. The method according to claim 24, wherein the inflammatory condition is related to tissue trauma or resulting from organ transplantations or other surgical operations.

36. The method according to claim 24, wherein said disease related to carbohydrate metabolism is selected from the group consisting of diabetes, atherosclerosis, vascular retinopathies, retinopathy, nephropathy, nephrotic syndrome, polyneuropathy, mononeuropathies, autonomic neuropathy, foot ulcers or joint problems.

37. The method according to claim 24, wherein said disease relating to aberrations in adipocyte differentiation or function or smooth muscle cell function is selected from the group consisting of atherosclerosis and obesity.

38. The method according to claim 24, wherein said vascular disease is selected from the group consisting of atheromatous ateriosclerosis, nonaatheromateous ateriosclerosis, ischemic heart disease, peripheral aterial occlusion, thromboangiitis obliterans (Buerger's disease), and Raynaud's disease and phenomenon.
